(19) United States
(12) Patent Application Publication
(10) Pub. No.: US 2018/0284093 A1
(43) Pub. Date: Oct. 4, 2018

(54) Trusted Food Traceability System and Method and Sensor Network

(71) Applicant: Innit International S.C.A., Luxembourg (LU)

(72) Inventors: Kevin Brown, Atherton, CA (US); Eugenio Minvielle, Hillsborough, CA (US)

(21) Appl. No.: 15/940,780

(22) Filed: Mar. 29, 2018

Related U.S. Application Data
(60) Provisional application No. 62/478,555, filed on Mar. 29, 2017.

(51) Int. Cl. G01N 33/02; H04L 9/06; H04L 29/08; G06Q 50/28; H04W 4/35

(52) U.S. Cl. CPC G01N 33/02; H04L 9/0637; H04W 4/35; G06Q 50/28; H04L 67/1042

(57) ABSTRACT

A food traceability and alert system utilizing blockchain or similar structures with cryptographic signatures, distributed sensors throughout the supply chain, and cloud infrastructure to provide trusted information from all states of the supply chain is disclosed.

### CROSS REFERENCE TO RELATED APPLICATIONS

This application claims the benefit of, and priority to, U.S. Provisional Patent Application Ser. No. 62/478,555, filed Mar. 29, 2017, entitled “Trusted Food Traceability System and Method and Sensor Network” the entire disclosure of which is hereby incorporated by reference.

### FIELD OF THE INVENTION

Embodiments of the present inventions relate generally to food traceability through a supply chain. More specifically, embodiments relate to a food traceability and alert system utilizing blockchain or similar structures with cryptographic signatures, distributed sensors throughout the supply chain, and cloud infrastructure to provide trusted information from all states of the supply chain. Embodiments of the present invention further relate to connecting that information to consumer kitchens.

### BACKGROUND OF THE INVENTION

Nutritional substances are traditionally grown (plants), raised (animals) or synthesized (synthetic compounds). Additionally, nutritional substances can be found in a wild, non-cultivated form, which can be caught or collected. While the collectors and creators of nutritional substances generally obtain and/or generate information about the source, history, caloric content and/or nutritional content of their products, they generally do not pass such information along to the users of their products. It would be desirable for such information be available to the consumers of nutritional substances, as well as all participants in the food and beverage industry—the nutritional substance supply system.

Consumers and activists are beginning to demand that the food and beverage industry offer products which include higher nutritional content, and/or at least provide information regarding nutritional content of such products, as well as traceability and information on sustainability, that is, information regarding the source, creation and other origin information for the nutritional substance. In fact, consumers are already willing to pay higher prices for higher nutritional content. This can be seen at high-end grocery stores which offer organic, non GMO, minimally processed, fresh, non-adulterated nutritional substances. Further, as societies and governments seek to improve their constituents' health and lower healthcare costs, incentives and/or mandates will be given to the food and beverage industry to track, maintain, and/or increase the nutritional content of nutritional substances they handle. Furthermore, as the technology advances it generates the need for the industry to operate into a new operating framework. There will be a need for an industry-wide solution to allow the management, traceability and recordkeeping of relevant origin and quality information of nutritional content across the entire cycle from creation to consumption. Traceability provides the means to identify and trace the history, distribution, location, and application of products, parts and materials. A traceability system follows, records and controls processes, from farm to fork. In order to manage the nutritional content of nutritional substances across the entire cycle from creation to consumption, the nutritional substance industry will need to identify, track, measure, estimate, preserve, transform, condition, and record nutritional content for nutritional substances. The most common problems in the supply chain of nutritional substances are fraud (substitution, tampering, misrepresentation), illegal production of unreported/unregulated nutritional substances (e.g. seafood), foodborne illness, and food recall/loss due to a abuse in the supply chain.

Of particular importance is the measurement, estimation, and tracking of changes to the nutritional content and quality parameters of a nutritional substance from creation to consumption. This information could be used, not only by the consumer in selecting particular nutritional substances to consume, but could be used by the other food and beverage industry participants, including creation, preservation, transformation, and conditioning, to make decisions on how to create, handle and process nutritional substances. These industries that operate across the supply chain are looking for new solutions to assist in traceability and recordkeeping. Additionally, those who sell nutritional substances to consumers, such as restaurants and grocery stores, could communicate perceived qualitative values of the nutritional substance in their efforts to market and position their nutritional substance products. Further, a determinant of price of the nutritional substance could be particular nutritional, organoleptic, or aesthetic values, and if changes to those values are perceived as desirable. For example, if a desirable value has been maintained, improved, or minimally degraded, it could be marketed as a premium product. Still further, a system allowing creators, preservers, transformers, and conditioners of nutritional substances to update labeling content to reflect the most current information about the nutritional substance would provide consumers with the information they need to make informed decisions regarding the nutritional substances they purchase and consume. Such information updates could include nutritional, organoleptic, or aesthetic values of the nutritional substance, and may further include information regarding the source, creation and other origin information for the nutritional substance. As such valuable information available to consumers and others in the food ecosystem becomes more robust and accessible, it is critical that such information be accurate and verifiable. Tracking and traceability are critical, however in today's environment, much information is shared and transmitted digitally and as such, has become susceptible to tampering, such as by hacking. Thus, a very significant need exists to provide trusted food traceability systems and methods.

### OBJECTS OF THE INVENTION

To address the signification need to provide trusted food traceability systems and methods, the inventors have developed and explored the potential for blockchain or similar technologies to change the nutritional substance information (e.g. nutritional, quality, origins) transfer in the food ecosystem. One of the key advantage of using blockchain against current systems is auditability and trust. Once the information is recorded in a blockchain, the information is immutable and can be referenced at any point in the future, allowing parties to verify that the information is reliable. Second, robust blockchains can take a variety of information without being restricted to a particular format, such as data from an inventory management system, batch or lot records for unique identification, GPS data for location information or cold chain data. As a result, blockchain systems can serve as middleware, allowing interoperability between different software platforms that may need to exchange information.

It is an object of the present inventions to provide a food traceability and alert system utilizing blockchain or similar structures with cryptographic signatures, distributed sensors throughout the supply chain, and cloud infrastructure to provide trusted and verifiable information from all states of the supply chain. It is a further object of the present inventions to connect such information to consumer kitchens.

In an object of the present invention is to allow for changes of nutritional, organoleptic, and/or aesthetic values of a nutritional substance (herein sometimes referred to as ΔN) to be tracked and degradation of said value to be tracked and minimized, and for such information to be verifiably tracked using blockchain or similar cryptographic signatures. In a further object, information regarding said changes or degradation, and information related to origin and creation of the nutritional substance, is collected, stored, and transmitted, from creation through consumption, including all phases of preservation, transformation, local storage and conditioning, and verifiably tracked using blockchain or similar cryptographic signatures.

In an object of the present invention, appliances and equipment are provided to track changes of nutritional, organoleptic, and/or aesthetic values of a nutritional substance, and to minimize and/or track degradation of said values, and/or collect, store, and/or transmit information regarding these changes or degradation, and information related to origin and creation of the nutritional substance, during local storage and conditioning of the nutritional substance, and verifiably tracked using blockchain or similar cryptographic signatures.

In a further object of the present invention, local storage and or conditioning of a nutritional substance is modified or adapted to maintain and/or minimize degradation of and/or improve nutritional, organoleptic, and/or aesthetic values of the nutritional substance, responsive to information sensed during said local storage regarding a nutritional, organoleptic, or aesthetic value of the nutritional substance, including information relating to the weight of the substance, and verifiably tracked using blockchain or similar cryptographic signatures.

In an object of the present invention, information collected by sensors of, or sensors communicating with, a local appliance (such as a local storage appliance), can collect all types of physical attribute data by sensing a nutritional substance, and that the nutritional substance can be identified and its current nutritional, organoleptic, and aesthetic state determined, by comparing the sensed data to a library of data for known nutritional substances at known nutritional, organoleptic, and aesthetic states, and further that the nutritional substance can be adaptively stored responsive to: its initial nutritional, organoleptic, or aesthetic state; consumer input received through a consumer interface of the local storage appliance related to a desired nutritional, organoleptic, or aesthetic state after local storage; and information sensed during local storage related to changes in the nutritional substance's nutritional, organoleptic, or aesthetic state, and wherein such information is verifiably tracked using blockchain or similar cryptographic signatures.

In an object of the present invention, information collected by sensors of, or sensors communicating with, a conditioning appliance, can collect all types of physical attribute data, by sensing a nutritional substance, and that the nutritional substance can be identified and its current nutritional, organoleptic, and aesthetic state determined and verifiably tracked using blockchain or similar cryptographic signatures.

### SUMMARY OF THE INVENTION

In an embodiment of the present invention, a system and method is provided relating to a food traceability and alert systems utilizing blockchain or similar structures with cryptographic signatures, distributed sensors throughout the supply chain, and cloud infrastructure to provide trusted information from all states of the supply chain. In another embodiment of the present invention, the food traceability and alert system connects such information to consumer kitchens.

In another embodiment, a system is provided for the tracking of changes of nutritional, organoleptic, and/or aesthetic values of a nutritional substance, wherein the system may collect, store, and transmit information regarding the changes of nutritional, organoleptic, and/or aesthetic values of the nutritional substance, and information related to origin and creation of the nutritional substance, from creation through consumption, including all phases of preservation, transformation, local storage and conditioning, and to the consumer, all verifiably tracked using blockchain or similar cryptographic signatures.

In one aspect, embodiments provide a method or system including sensors configured to measure ΔN of a nutritional substance or food handling environment at any stage of the supply chain, including creation, transportation, preservation, manufacturing, distribution, retail, or consumer homes.

In some embodiments, a distributed ledger or database is provided, with data stored in three or more locations, to store sensor measurements of nutritional substances or associated environments for storage, processing, conditioning, retail, and the like.

In some embodiments a processor is provided, based in the cloud or elsewhere, that makes calculations about the status of nutritional substances based on sensor data. A database may be provided that associates the status and ownership of the nutritional substance with various parties in the supply chain, including the end consumer who has purchased the item.

In another aspect, a cryptographic mechanism is provided, such as blockchain, Merkle trees or other cryptographically signed data structures, to verify the authenticity and source of data associated with a nutritional substance, and to provide a consensus mechanism for determining the valid value of a data element.

Further, a reporting mechanism may be provided, such as email, SMS, Web page, application, or embedded appliance software functionality, that accesses the database to provide information on the nutritional substance. In some embodiments, the reporting mechanism assesses the information based on at least two measurements of the nutritional substance.

In an even further embodiment, an alerting network is provided which can communicate with supply chain participants or consumers, to identify nutritional substance items and associated ΔN, organoleptic status, or food-safety status; such as for example, the potential presence of chemical or biological contaminants.

In another aspect, a consumer sensor system is provided, comprised of a mobile device, smart appliance, or modular sensor node that is configured to identify nutritional substances via dynamic information identifier, bar-code, identification number, optical recognition, computer vision, spectral recognition, electronic recognition such as RFID, NFC, or any other electromagnetic tag, product packaging or labels, or molecular composition of a nutritional substance or associated additives.

In another embodiment, a consumer sensor system is provided, comprised of a mobile device, smart appliance, or modular sensor node that can sense ΔN of food substances, including food safety, freshness or organoleptic status. Sensing may be accomplished by means including optical recognition, computer vision, spectral recognition, gas sensing, electronic recognition such as RFID, NFC, or any other electromagnetic tag, active product packaging or labels, sensors for reading packages or electromagnetic tags, or molecular composition of a nutritional substance or associated additives.

In some embodiments the system can associate products with a consumer via multiple methods, including but not limited to: bar-code scan, receipt scan, loyalty card integration, food recognition, voice input, or any other method for food purchases to be manually or automatically logged.

In some embodiments, the Blockchain entries may include a geo-location tag, and associated lookup of address and associated location identity (such as for example, store name, farm location, mobile transportation reading, and the like.)

Other advantages and features will become apparent from the following description and claims. It should be understood that the description and specific examples are intended for purposes of illustration only and not intended to limit the scope of the present disclosure.

### BRIEF DESCRIPTION OF THE DRAWINGS

The accompanying drawings, which are incorporated in and constitute a part of this specification, exemplify the embodiments of the present invention and, together with the description, serve to explain and illustrate principles of the invention. The drawings are intended to illustrate major features of the exemplary embodiments in a diagrammatic manner. The drawings are not intended to depict every feature of actual embodiments nor relative dimensions of the depicted elements, and are not drawn to scale.

FIG. 1 illustrates a schematic functional block diagram of a nutritional substance supply system with integrated blockchain (or similar cryptographic signature system) traceability and alert network according to embodiments of the present invention.

FIG. 2 shows a graph representing a value of a nutritional substance which changes according to a change of condition for the nutritional substance.

FIG. 3 shows a graph representing a value of a nutritional substance which changes according to changes in multiple conditions for the nutritional substance.

FIG. 4 shows a graph representing a value of a nutritional substance which changes according to changes in multiple conditions for the nutritional substance.

FIG. 5 depicts the flows of products and information within a nutritional substance supply chain, including information storage in a distributed ledger blockchain-like infrastructure. Information flows through one or more cloud servers or computers, and can also flow into the consumer's home.

In the drawings, the same reference numbers and any acronyms identify elements or acts with the same or similar structure or functionality for ease of understanding and convenience. To easily identify the discussion of any particular element or act, the most significant digit or digits in a reference number refer to the Figure number in which that element is first introduced.

### DETAILED DESCRIPTION OF THE INVENTION

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other obvious features not described in detail herein. Additionally, some well-known structures or functions may not be shown or described in detail below, so as to avoid unnecessarily obscuring the relevant description.

The terminology used below is to be interpreted in its broadest reasonable manner, even though it is being used in conjunction with a detailed description of certain specific examples of the invention. Indeed, certain terms may even be emphasized below; however, any terminology intended to be interpreted in any restricted manner will be overtly and specifically defined as such in this Detailed Description section.

The following discussion provides a brief, general description of a representative environment in which the invention can be implemented. Although not required, aspects of the invention may be described below in the general context of computer-executable instructions, such as routines executed by a general-purpose data processing device (e.g., a server computer or a personal computer). Those skilled in the relevant art will appreciate that the invention can be practiced with other communications, data processing, or computer system configurations, including: wireless devices, Internet appliances, hand-held devices (including personal digital assistants (PDAs)), wearable computers, all manner of cellular or mobile phones, multi-processor systems, microprocessor-based or programmable consumer electronics, set-top boxes, network PCs, mini-computers, mainframe computers, and the like. Indeed, the terms “controller,” “computer,” “server,” and the like are used interchangeably herein, and may refer to any of the above devices and systems.

While aspects of the invention, such as certain functions, are described as being performed exclusively on a single device, the invention can also be practiced in distributed environments where functions or modules are shared among disparate processing devices. The disparate processing devices are linked through a communications network, such as a Local Area Network (LAN), Wide Area Network (WAN), or the Internet. In a distributed computing environment, program modules may be located in both local and remote memory storage devices.

Aspects of the invention may be stored or distributed on tangible computer-readable media, including magnetically or optically readable computer discs, hard-wired or preprogrammed chips (e.g., EEPROM semiconductor chips), nanotechnology memory, biological memory, or other data storage media. Alternatively, computer implemented instructions, data structures, screen displays, and other data related to the invention may be distributed over the Internet or over other networks (including wireless networks), on a propagated signal on a propagation medium (e.g., an electromagnetic wave(s), a sound wave, etc.) over a period of time. In some implementations, the data may be provided on any analog or digital network (packet switched, circuit switched, or other scheme).

In some instances, the interconnection between modules is the internet, allowing the modules (with, for example, WiFi capability) to access web content offered through various web servers. The network may be any type of cellular, IP-based or converged telecommunications network, including but not limited to Global System for Mobile Communications (GSM), Time Division Multiple Access (TDMA), Code Division Multiple Access (CDMA), Orthogonal Frequency Division Multiple Access (OFDM), General Packet Radio Service (GPRS), Enhanced Data GSM Environment (EDGE), Advanced Mobile Phone System (AMPS), Worldwide Interoperability for Microwave Access (WiMAX), Universal Mobile Telecommunications System (UMTS), Evolution-Data Optimized (EVDO), Long Term Evolution (LTE), Ultra Mobile Broadband (UMB), Voice over Internet Protocol (VoIP), Unlicensed Mobile Access (UMA), etc.

The modules in the systems can be understood to be integrated in some instances and in particular embodiments, only particular modules may be interconnected.

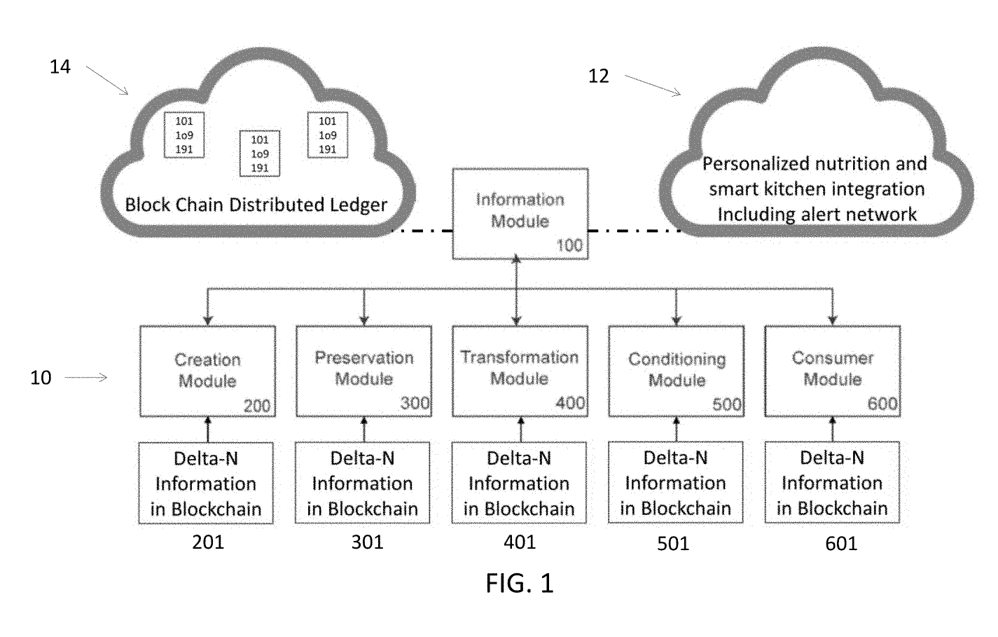

FIG. 1 shows a schematic of block diagram of the components of a nutritional substance supply industry 10, with a trusted food traceability and optional alert system according to one embodiment. It should be understood that this could be the food and beverage ecosystem for human consumption, but it could also be the feed industry for animal consumption, such as the pet food industry. A goal of the present invention for nutritional substance industry 10 is to facilitate the transfer of traceability of quality and nutrition and sustainability information in a trusted and verifiable manner across the different industries participants of a given food ecosystem. This will enable the food industry to trace the change in nutritional, organoleptic and/or aesthetic values of nutritional substances, collectively and individually also referred to herein as ΔN, through their creation, preservation, transformation, conditioning and consumption. In this embodiment, a block chain, or similar system using cryptographic signatures, traceability and alert network 12 is integrated into the nutritional supply system 10. Information and transactions of nutritional substances between supply chain participants are bundled together into blocks, which are then linked to the previous block, forming a chain. As illustrated in FIG. 1, ΔN information in blockchain format may be provided at one, more or all of the nutritional system modules. The benefit of using blockchain 14 is due to the fact that it is an immutable, auditable platform to record data among parties participating in the different modules described, with the promise of reduced traceability time and better supply chain collaboration. Blockchain provides a peer-to-peer network in which all the authorized players are able to fix transactions and validate them in real time. In addition, blockchain offers a clear value proposition in food safety, as events could be traced back to identify risk factors and uniquely identified products could be pulled in a targeted recall instead of a blanket recall. Furthermore, the auditability of a blockchain offers the promise of being able to meet consumer, shareholder and buyer demand for increased supply chain transparency, should data be posted publicly or shared among selected participants.

The use of blockchain also increases the trust between the participants of the supply chain of the nutritional substances by combining auditability and transparency. Every party can confirm the validity and authenticity of the blockchain and efficiently read or add new information. However, no one can modify or delete records without the permission of other participants. The system enables the detailed traceability of assets, under the conditions that they remain the responsible party/owner. Furthermore, permissioned blockchains require a package of permissions (access and identity management) that prevent fraud and/or access to other's party confidential information. New members must have an invitation and be validated to the blockchain system.

While the nutritional substance industry 10 can be composed of many companies or businesses, it can also be integrated into combinations of business serving many roles, or can be one business or even an individual. Since ΔN is a measure of the change in a value of a nutritional substance, knowledge of a prior value (or state) of a nutritional substance and the ΔN value will provide knowledge of the changed value (or state) of a nutritional substance, and can further provide the ability to estimate a change in value (or state). The ΔN value may be represented or displayed to a consumer as a per unit weight (e.g., ΔN per ounce, or ΔN per gram) format or value, may be displayed as a graph showing the change of the in the nutritional substance over time or in various other formats that would demonstrate a change in a ΔN. For example, a consumer may be presented with a graph showing the historical or prospective change in the nutritional, organoleptic and/or aesthetic values of the nutritional substance, over time, cooking temperatures, or other choices or attributes. This presents a continuum to the consumer of how ΔN may change with the change in various factors including time and cooking temperature. Color codes, visualizations, animations, charts, scores, numeric values, auditory interactions, sounds, haptic feedback and other communication means may be used to convey ΔN.

The ΔN value may also represent a comparison between the gold standard or average for a nutritional substance, and a particular or actual nutritional substance a consumer is considering purchasing. Accordingly, the attributes of a particular nutritional substance can be compared to the expected or optimal attributes of that type or category of nutritional substance. This allows a consumer to make more informed choices about the nutritional value of a substance a consumer is contemplating purchasing, or make informed decisions about preparation of the nutritional substance. For example, ΔN may represent a difference in the vitamin C content between on optimal orange that is picked when ripe from the vine, and an actual orange that a consumer is considering purchasing. In this example, if the consumer's orange was picked from the vine early, it may have both different surface physical characteristics that may be detectable by the sensors and methods described herein, and different vitamin C content. A database comprised of blockchain distributed ledgers as described herein may include information regarding the physical attributes of an orange, and possibly how those factors correlate to the vitamin C content and other nutritional information. The values recorded in the blockchain distributed ledger are uploaded by the entity creating and/or storing and/or transporting, and/or distributing and/or transforming and/or selling the nutritional substance(s). This is accomplished by embedding information collected by sensors or analytical instruments into the distributed ledger and authenticated by a unique digital cryptographic signature. This information is then stored in three or more locations, broadcasted and distributed through the network by storing this information in the blockchain.

Accordingly, the systems disclosed herein may be able to determine the difference in vitamin C between a specific orange and the average vitamin C in oranges or the optimal vitamin C of, for example, an orange just picked from the vine when ripe. Accordingly, ripeness of tomatoes, water content, vitamin content, and other nutritional, organoleptic and/or aesthetic values may be compared for a specific, actual item a consumer is considering purchasing to the average or gold standard for that item. Accordingly, by accessing the information available in the distributed ledger for that batch of oranges or that specific orange, a consumer may then discern whether that particular item is providing at least an average or optimal nutrient, organoleptic and/or aesthetic value.

These differences may be presented in absolute value, for instance the difference in vitamin C, as a per unit weight value, as a graph comparing the present item versus an average curve for that specific item, or may be presented as a difference in nutritional content per unit price. For example, certain oranges or farmer's market produce may claim to have higher nutritional content because they are fresher or were harvested from the vines/roots closer in time to when the fruit ripened, leading to a higher nutritional content. However, these fruits tend to be higher in price, and accordingly, the system may be utilized to determine whether higher priced fruits are actually worth the higher price, and the amount of nutritional value gained per dollar difference.

In other examples, ΔN may represent the difference between the nutritional content of different subtypes of a broader category of nutritional substance. For instance, wild caught salmon is claimed to have up to 10 times greater omega three content than farm raised salmon. Accordingly, the present system could compare the nutritional content of a specific farm raised salmon to different types of wild caught salmon to determine the difference or ΔN in the omega three values. As described herein, this difference may be presented as an absolute value based on weight, an omega three difference per dollar, a per unit weight difference, or a graph indicating difference points including, average, optimum, and the current value of the fish on the graph.

By making the block chain data available to the consumer at the retailer of nutritional substances, consumers could make informed choices based on quantitative data about whether and how much more nutritious more expensive fruit may be actually worth to the consumer. Other estimates related to firmness, freshness, flavor, and texture could also be used to determine or analyze prices. For example, the retailer could in principle have a kiosk where the product information can be scanned via optical means through barcode, QRC, machine vision or any other means for optical identification, or by electronic means such as RFID or NFC tags, or by chemical or mechanical tags. Moreover, this technology can also help the retailer to withdraw product that has been recalled, or to re-price the item based on feedback and ratings from customers or other entities. The system can automatically alert the retailer that a recall of a given batch of nutritional substance has taken place, so the risk to consumers is minimized by removing the nutritional substances in question from the shelf, or by warning the clerk (or consumer using an automatic kiosk) that the item has been recalled during the checkout process.

Referring again to FIG. 1, module 200 is the creation module. This can be a system, organization, or individual which creates and/or originates nutritional substances. Examples of this module include a farm which grows produce; a ranch which raises beef; an aquaculture farm for growing shrimp; a factory that synthesizes nutritional compounds; a collector of wild truffles; or a deep sea crab trawler. In this module, the entity that created or originated the nutritional substances can record quality, origin, nutritional content, creation dates or other information pertinent to the nutritional substance directly into a new blockchain distributed ledger 201 by embedding information collected by manual data entry means, through sensors or analytical instruments into the distributed ledger, accessed through a unique digital cryptographic signature.

Preservation module 300, described in co-pending application U.S. Ser. No. 13/888,353, titled “Preservation System for Nutritional Substances”, and incorporated in its entirety by reference herein, is a preservation system for storing, preserving and protecting the nutritional substances created by creation module 200. Once the nutritional substance has been created, generally, it will need to be packaged in some manner for its transition to other modules in the nutritional substances industry 10. While preservation module 300 is shown in a particular position in the nutritional substance industry 10, following the creation module 200, it should be understood that the preservation module 300 actually can be placed anywhere nutritional substances need to be stored and preserved during their transition from creation to consumption. For instance, preservation module 300 may be placed after transformation module 400 but prior to conditioning module 500, to store the nutritional substance either in a retail establishment or in a consumer's household. This storage may include on a shelf, in a refrigerator, or in a freezer at a consumer residence, restaurant, grocery store or other retail establishment. It is understood that a nutritional substance may experience more than one preservation event, and that such preservation events may include the local storage of the nutritional substance, such as by a consumer prior to conditioning or consumption in addition to storage along the food processing chain.

A specific aspect of the present invention in achieving its goal related to ΔN information is to provide a system that tracks ΔN information during local storage or local preservation of a nutritional substance by a consumer. In this module, the entity that is preserving the nutritional substances can add information on the distributed ledger information gather through data entry or sensors such as temperature, relative humidity of the environment the nutritional substance was exposed to, or any measurements pertaining to the nutritional substance made by optical means (such as NIR, visible light, Raman spectroscopy), or obtained by reading chemical, genetic, electronic or mechanical tags. This information is added directly into a new blockchain 301 by embedding information collected by sensors or analytical instruments into the distributed ledger, accessed through a unique digital cryptographic signature.

It is understood that a nutritional substance may experience more than one preservation event, and that such preservation events may include any known form of local storage or local preservation of a nutritional substance prior to conditioning and/or consumption, hereinafter referred to as local storage. Such local storage may take many forms, such as the storage of refrigerated items in a refrigerator, the storage of frozen items in a freezer, the storage of wine bottles in a wine-rack, the storage of canned or dry goods in a pantry, the storage of bread in a bread drawer, the storage of fruit in a counter top tray, and any other form of local nutritional substance storage known to those skilled in the art. It is understood that the present inventions include the local storage of consumable items such as medicaments, for example, medicaments stored in a refrigerator, medicaments stored in a medicine cabinet, or medicaments stored in any other known fashion.

More specifically, blockchain 14 or similar structures with cryptographic signatures, distributed sensors throughout the supply chain, and cloud infrastructure provide trusted information from all states of the supply chain. An alert network 12 may also be provided for personalized nutrition and smart kitchen integration which connects verifiable information to consumer kitchens.

Sensors are configured to identify nutritional substances, while measuring ΔN of the nutritional substance or food handling environment at any stage of the supply chain, including creation, transportation, preservation, manufacturing, distribution, retail, or consumer homes.

Local storage according to the present invention can be enabled by local storage environments according to the present invention, such as a refrigerator, drawer, cabinet, portable cooler, and any other type of storage environment, wherein the local storage environment is provided with the same capabilities as the preservation module. In addition; local storage according to the present invention can be enabled by local storage containers according to the present invention, such as storage bags, trays, resalable storage-ware, jars, boxes, bottles, and any other type of storage environment, wherein the local storage container is provided with the same capabilities as the preservation module. A reporting mechanism may be used, such as email, SMS, Web page, application, or embedded appliance software functionality, that accesses the database to provide information on the nutritional substance. In some embodiments, the reporting mechanism assesses the information based on at least two measurements of the nutritional substance. To integrate with the consumer and provide trusted integrity to the system, an alerting network is provided which can communicate with supply chain participants or consumers, to identify nutritional substance items and associated ΔN, organoleptic status, or food-safety status; such as for example, the potential presence of chemical or biological contaminants.

Consumer sensor systems may be employed. In one embodiment, the consumer sensor system is comprised of a mobile device, smart appliance, or modular sensor node that is configured to identify nutritional substances via dynamic information identifier, bar-code, identification number, optical recognition, computer vision, spectral recognition, electronic recognition such as RFID, NFC, or any other electromagnetic tag, product packaging or labels, or molecular composition of a nutritional substance or associated additives.

Another consumer sensor system may be provided which is comprised of a mobile device, smart appliance, or modular sensor node that can sense ΔN of food substances, including food safety, freshness or organoleptic status. Sensing may be accomplished by any suitable means, such as but not limited to optical recognition, computer vision, spectral recognition, gas sensing, electronic recognition such as RFID, NFC, or any other electromagnetic tag, active product packaging or labels, sensors for reading packages or electromagnetic tags, or molecular composition of a nutritional substance or associated additives.

In some embodiments the system can associate products with a consumer via multiple methods, including but not limited to: bar-code scan, receipt scan, loyalty card integration, food recognition, voice input, or any other method for food purchases to be manually or automatically logged.

In the context of a smart kitchen, optical, chemical, electromagnetic (e.g. RFID, NFC), mechanical or genetic sensors can be embedded in the local storage in the form of appliance (e.g. refrigerator) or pantry or sensor accessor that can detect and identify a specific nutritional substance in an active product packaging with labels. The sensors embedded in or placed near the appliance or pantry are connected to a cloud based blockchain database. In the case of a recall of a batch of nutritional substances due to contamination at any step in the supply chain, the sensors of the local storage unit can detect a nutritional substance item and trigger an alarm to the consumer. The alarm can be sent in the form of a notification in a mobile device app, which indicates to the consumer that a recall for that specific items has been issued, and instructions as to how to proceed in discarding, disposing or returning the item in question. Additionally, a coupon or credit can be sent to the consumer once the consumer confirms that the nutritional substance has been discarded or that the nutritional substance is no longer available for consumption. In this manner, the blockchain distributed ledger system adds value not only to the participants of the nutritional substance supply chain but also to the consumer by preventing the consumption of a contaminated nutritional substance. In addition, the consumer can be automatically compensated for the inconvenience of having to dispose of an item, generating more trust based on a reliable information blockchain system.

Transformation module 400 is a nutritional substance processing system, such as a manufacturer who processes raw materials such as grains into breakfast cereals. Transformation module 400 could also be a ready-to-eat dinner manufacturer who receives the components, or ingredients, also referred to herein as component nutritional substances, for a ready-to-eat dinner from preservation module 300 and prepares them into a frozen dinner. While transformation module 400 is depicted as one module, it will be understood that nutritional substances may be transformed by a number of transformation modules 400 on their path to consumption. Information regarding transformation may be added directly into a new blockchain 401 by embedding information collected by sensors or analytical instruments, or by entry by the transformer, into the distributed ledger, accessed through a unique digital cryptographic signature.

Conditioning module 500 is a consumer preparation system for preparing the nutritional substance immediately before consumption by the consumer. Conditioning module 500 can be a microwave oven, a blender, a toaster, a convection oven, toaster oven, a cook, etc. It can also be systems used by commercial establishments to prepare nutritional substance for consumers such as a restaurant, an espresso maker, pizza oven, and other devices located at businesses which provide nutritional substances to consumers. Such nutritional substances could be for consumption at the business or for the consumer to take out from the business. Conditioning module 500 can also be a combination of any of these devices used to prepare nutritional substances for consumption by consumers. Conditioning information may be added directly into a new blockchain 501 by embedding information collected by sensors, analytical instruments or by a user, into the distributed ledger, accessed through a unique digital cryptographic signature.

Consumer module 600 collects information from the living entity which consumes the nutritional substance which has passed through the various modules from creation to consumption. The consumer can be a human being, but could also be an animal, such as pets, zoo animals and livestock, which are they themselves nutritional substances for other consumption chains. Consumers could also be plant life which consumes nutritional substances to grow. Consumer information may be added directly into a new blockchain 601 by embedding information collected by sensors, analytical instruments, or by consumer entry, into the distributed ledger, accessed through a unique digital cryptographic signature.

Information module 100 receives and transmits information regarding a nutritional substance between each of the modules in the nutritional substance industry 10 including, the creation module 200, the preservation module 300, the transformation module 400, the conditioning module 500, and the consumer module 600. The nutritional substance information module 100 can be an interconnecting information transmission system which allows the transmission of information between various modules. Information module 100 contains a database, also referred to herein as a dynamic nutritional value database, where the information regarding the nutritional substance resides, particularly ΔN for the nutritional substance. A distributed ledger or database 14, (such as but not limited to a blockchain distributed ledger) with data stored in three or more locations, stores sensor measurements of nutritional substances or associated environments for storage, processing, conditioning, retail, and the like.

A processor (not shown) is provided, based in the cloud or elsewhere, that makes calculations about the status of nutritional substances based on sensor data. This is achieved by communication between the sensors and the processor. The processor is preferably (but not necessarily) hosted in a cloud (i.e. the processing center). The processing center in the cloud is composed of central databases of conditioning sessions and analytics and may include self—learning algorithms and Artificial Intelligence (AI), or other statistical and logical analyses of patters on data to uncover meaningful patterns and information.

To provide a trusted and verifiable system, a cryptographic mechanism is provided, such as blockchain, Merkle trees or other cryptographically signed data structures, to verify the authenticity and source of data associated with a nutritional substance, and to provide a consensus mechanism for determining the valid value of a data element.

A database may be provided such that it associates the status and ownership of the nutritional substance with various parties in the supply chain, including the end consumer who has purchased the item. Cryptocurrency transactions may be associated with the operations of the system. Information module 100 may also contain a massive database of physical attributes of known nutritional substances at known nutritional, organoleptic, and aesthetic states, also referred to herein as nutritional substance attribute library, which can be utilized for determining the identity and current nutritional, organoleptic, and aesthetic state of a nutritional substance. Information module 100 can be connected to the other modules by a variety of communication systems, such as paper, computer networks, the internet and telecommunication systems, such as wireless telecommunication systems. In a system capable of receiving and processing real time consumer feedback and updates regarding changes in the nutritional, organoleptic, and/or aesthetic value of nutritional substances, or ΔN, consumers can even play a role in updating a dynamic nutritional value database with observed or measured information about the nutritional substances they have purchased and/or prepared for consumption, so that the information is available and useful to others in the nutritional substance supply system, such as through reports reflecting the consumer input or through modification of ΔN. Mobile sensors, sensor accessories, or sensors embedded in appliances may be used to collect data about nutritional substances.

In an embodiment of the present invention, such consumer feedback and updates related to ΔN information are provided during the local storage of a nutritional substance. For example, a consumer may report that a basket of berries was over-ripe and spoiled shortly after purchase; other consumers may consult individual or aggregated reports to choose a retail outlet or brand with fresher nutritional substances. In a preferred embodiment, such consumer feedback and updates related to ΔN information are obtained through, or provided by, local storage environments, local storage containers, and local storage coupons according to the present invention.

In some embodiments of the present invention, consumer feedback and updates regarding ΔN information may be obtainable from appliances that include the ability to display ΔN information, including ΔN information calculated based on a sensed physical attribute of the nutritional substance. The ΔN value may be calculated, represented, or displayed to a consumer as a per unit weight (e.g., ΔN per ounce, or ΔN per gram) format or value. The ΔN value may also represent a comparison between the gold standard or average for a particular nutritional substance, and a particular nutritional substance. For example, a graphic may be displayed portraying a value between 1 and 100 to communicate a level of freshness, remaining time until expiration, or a summary statistic of multiple ΔN values. Accordingly, the attributes of a particular nutritional substance can be compared to the expected or optimal attributes of that type or category of nutritional substance. This allows a consumer to make more informed choices about the nutritional value of a substance a consumer is contemplating purchasing or make informed decisions about preparation of the nutritional substance. For instance, a scale or other weight measurement device, alone or incorporated into another appliance may be provided with the ability to detect the weight and calculate a ΔN based on a current weight of the nutritional substance and be interconnected to nutritional substance information module 100. Accordingly, a standalone scale may be provided with the ability to detect the weight of a nutritional substance, and display ΔN information to the consumer based on the current weight of the nutritional substance and provide that information to the nutritional substance information module 100. Accordingly, this information may be integrated with the other modules including conditioning module 500 and preservation module 300. Additionally, a scale or other weight sensor may be integrated into a variety of other appliances to provide the ability to display ΔN information to the consumer based on the current weight of a nutritional substance. Accordingly, a weight sensor may be integrated into a storage container, shelf, drawer, refrigerator, microwave, smart oven, oven, conditioner, local storage container, or any other appliances that store, condition or otherwise interact with nutritional substances. An example of an electronic scale is described in, for example, U.S. Pat. No. 6,538,215, issued on Mar. 25, 2003, titled Programmable Digital Scale, which is incorporated by reference herein in its entirety.

In some embodiments nutritional substances may be identified by detection of a nutritional substance's optical characteristics. For example, various products are available capable using optical technology to visually identify produce and other nutritional substances, and various other items. For example, an automated optical fruit recognition system developed by Fraunhofer is capable of detecting and identifying various produce optically as described by an article titled “Automated Fruit Recognition” available at http://www.isob.fraunhofer.de/servlet/is/33328/ which is incorporated by reference herein in its entirety. Accordingly, the Fraunhofer system may be utilized to determine the identity of a nutritional substance by utilizing optical data detected from the nutritional substance. Accordingly, a user could then utilize their mobile phone or other devices with optical sensors to identify nutritional substances. Additionally, an optical object recognition system is disclosed in U.S. Pat. No. 6,310,964 that is described as capable of identifying produce and is incorporated herein by reference in its entirety.

The information pertaining to the nutritional substance that can be encrypted and stored into the blockchain distributed ledger 14 can be the change of nutritional, organoleptic or aesthetic value as a function of conditions such as time, temperature and/or exposure.

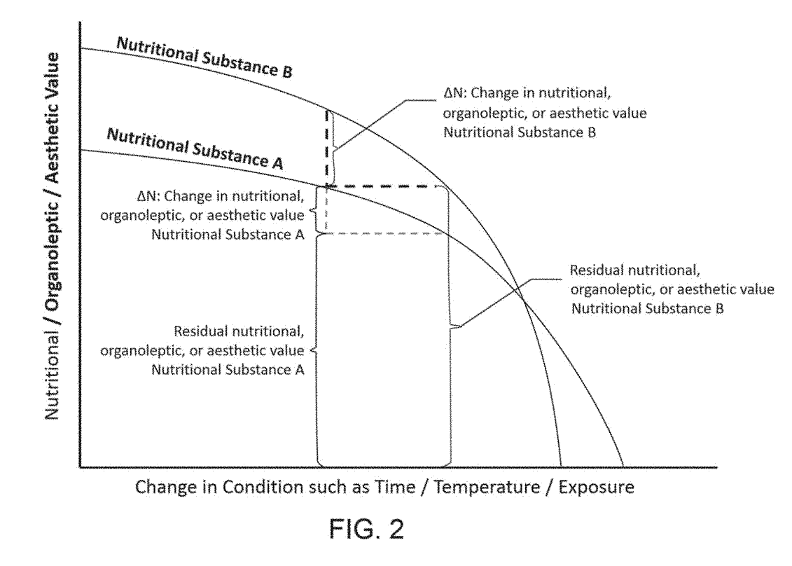

FIG. 2 is a graph showing the function of how a nutritional, organoleptic, or aesthetic value of a nutritional substance varies over the change in a condition of the nutritional substance. Plotted on the vertical axis of this graph can be either the nutritional value, organoleptic value, or even the aesthetic value of a nutritional substance. Plotted on the horizontal axis can be the change in condition of the nutritional substance over a variable such as time, temperature, location, and/or exposure to environmental conditions. This exposure to environmental conditions can include: exposure to air, including the air pressure and partial pressures of oxygen, carbon dioxide, ethylene, water, or ozone; airborne chemicals, pollutants, mold, allergens, dust, smoke, carcinogens, radioactive isotopes, or combustion byproducts; exposure to moisture; exposure to energy such as mechanical impact, mechanical vibration, irradiation, heat, or sunlight; or exposure to materials such as packaging. The function plotted as nutritional substance A could show a ΔN for milk, such as the degradation of a nutritional value of milk over time. Any point on this curve can be compared to another point on the same curve to measure and/or describe the change in nutritional value, or the ΔN, of nutritional substance A. The plot of the degradation in the same nutritional value of nutritional substance B, also milk, describes the change in nutritional value, or the ΔN, of nutritional substance B, a nutritional substance which starts out with a higher nutritional value than nutritional substance A, but degrades over time more quickly than nutritional substance A.

In this example, where nutritional substance A and nutritional substance B are milk, this ΔN information regarding the nutritional substance degradation profile of each milk could be used by the consumer in the selection and/or consumption of the milk. If the consumer has this information at time zero when selecting a milk product for purchase, the consumer could consider when the consumer plans to consume the milk, and whether that is on one occasion or multiple occasions. For example, if the consumer planned to consume the milk prior to the point when the curve represented by nutritional substance B crosses the curve represented by nutritional substance A, then the consumer should choose the milk represented by nutritional substance B because it has a higher nutritional value until it crosses the curve represented by nutritional substance A. However, if the consumer expects to consume at least some of the milk at a point in time after the time when the curve represented by nutritional substance B crosses the curve represented by nutritional substance A, then the consumer might choose to select the milk represented by the nutritional substance A, even though milk represented by nutritional substance A has a lower nutritional value than the milk represented by nutritional substance B at an earlier time. This change to a desired nutritional value in a nutritional substance over a change in a condition of the nutritional substance described in FIG. 2 can be measured and/or controlled throughout nutritional substance supply system 10 in FIG. 1. This example demonstrates how dynamically generated information regarding a ΔN of a nutritional substance, in this case a change in nutritional value of milk, can be used to understand a rate at which that nutritional value changes or degrades; when that nutritional value expires; and a residual nutritional value of the nutritional substance over a change in a condition of the nutritional substance, in this example a change in time. This ΔN information could further be used to determine a best consumption date for nutritional substance A and B, which could be different from each other depending upon the dynamically generated information generated for each.

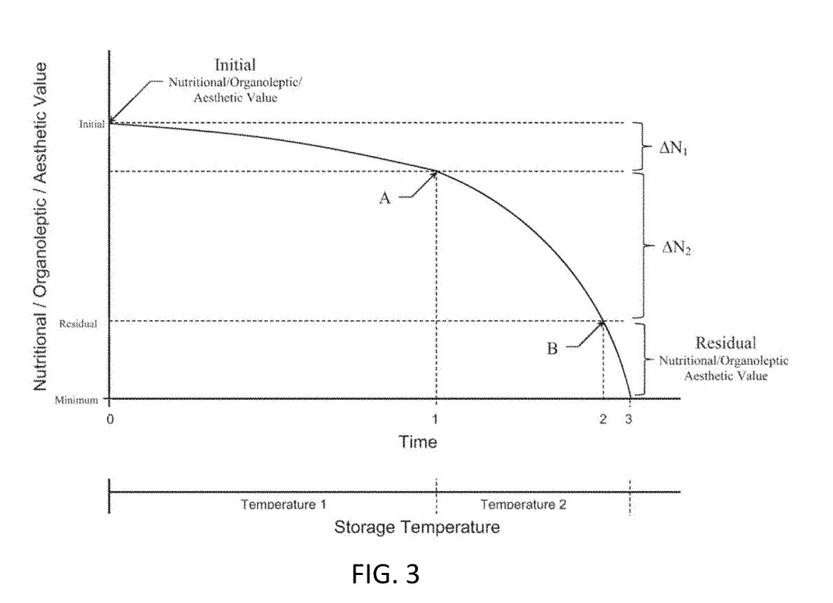

FIG. 3 is a graph showing the function of how a nutritional, organoleptic, or aesthetic value of a nutritional substance varies over a change in time and a change in a second condition, for instance the storage temperature of the nutritional substance or also may include an exposure or technology type change as illustrated. It is understood that change in time and change in storage temperature are offered by way of example and are in no way limiting to the types of condition changes (i.e. exposure, time and technology) to which the present inventions may be applied. As an example, the change in a nutritional property of milk is shown over a period of time including its preservation at the supermarket and a subsequent period of time including its local storage in a consumer's refrigerator, which is a local storage environment according to the present invention. The graph shows that the milk is preserved at a first temperature, Temperature 1, for a first period of time indicated as 0 to 1, while at the supermarket. The milk is purchased by a consumer at time 1, and subsequently stored at a second temperature, Temperature 2, for a second period of time indicated as 1 to 3, during local storage in the refrigerator, which is a local storage environment according to the present invention. It is noted that Temperature 2 is greater than Temperature 1, and accordingly the shape of the graph changes at point A when the milk is taken from Temperature 1 and stored at Temperature 2. As in the preservation module, the local storage environment can identify the milk stored within it by reading or scanning its dynamic information identifier (or by the consumer entering it), can communicate with the nutritional substance information module, and accordingly can determine the milk's ΔN prior to placement within the refrigerator, and continue to track the milk's ΔN while in the refrigerator. The refrigerator is provided with a consumer interface, such as a screen, keyboard, sound system, or any known consumer interface. The consumer interface enables the refrigerator to communicate to the consumer that it contains the particular carton of milk, information related to ΔN, including current nutritional, organoleptic, and aesthetic values of the milk, and when the milk will reach a minimum acceptable nutritional, organoleptic, or aesthetic value, indicated by “Minimum” on the vertical axis of the graph. The minimum acceptable values may be automatically provided by the information module, may be provided by the consumer through the consumer interface, or may be the higher of the two values. In this case the consumer can see how the nutritional value of the milk has degraded prior to purchasing it and can continue to see how the nutritional value degrades during local storage after its purchase, and when it will reach its minimum acceptable nutritional value. For example, at the time indicated as 2, the consumer can determine the residual nutritional value of the milk, corresponding to point B and “Residual” on the vertical axis of the graph. Further, the consumer can determine the milk's nutritional value will reach a minimum acceptable level at time 3, as indicated by “Minimum” on the vertical axis of the graph, thus knowing the window of time in which the milk will maintain an acceptable nutritional level, as indicated by time 1 to 3. Further, the refrigerator can notify the consumer through its consumer interface when the milk's nutritional value has reached or fallen below the minimum acceptable value.

In fact, if the consumer knows the internal temperature of his own refrigerator prior to purchasing the milk, he can predict the degradation of nutritional value of the milk that will occur after he purchases it and locally stores it in his refrigerator, thus knowing the window of time in which it will maintain an acceptable nutritional level, as indicated by time 1 to 3. For example, the consumer may utilize an application on his smartphone to store, or even monitor, the internal temperature of his refrigerator. When he goes to the supermarket, he could scan the milk's dynamic information identifier with his smartphone, and the application can communicate with the nutritional substance information module to determine a current ΔN and predict the ΔN of the milk when stored in his refrigerator. Further, the consumer may utilize such an application on his smartphone to store, or even monitor, the internal conditions of various local storage environments, local storage containers, and local storage coupons. In this way, when he goes to the supermarket, he can scan the dynamic information identifier of a wide variety of nutritional substances with his smartphone, and the application can communicate with the nutritional substance information module to determine a current ΔN and predict the ΔN of the nutritional substance when stored in the corresponding local storage environment or local storage container. In other embodiments, the consumer may place the milk, on a scale or other weight measurement device that allows the consumer to determine the current ΔN based on the weight of the milk left in the carton and the dynamic information identifier. For instance, the scale may have its own reader, or may be wirelessly connected to a smartphone that reads the identifier, and sends the data to a remote server or directly to the scale to combine with the weight data to determine the current ΔN.

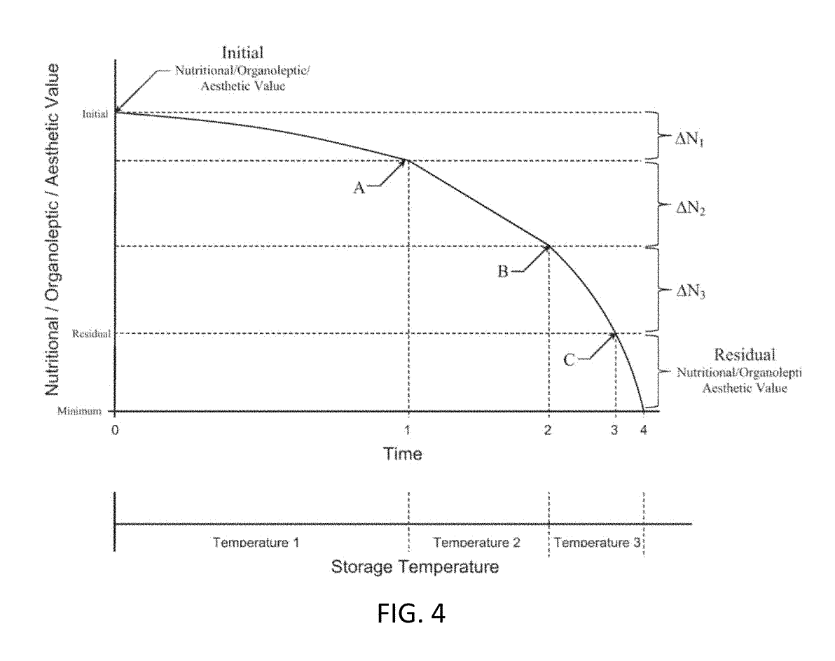

FIG. 4 is a graph showing the function of how a nutritional, organoleptic, or aesthetic value of a nutritional substance varies over a change in time and multiple changes in a second condition, including the storage temperature of the nutritional substance or the exposure or technology type. It is understood that change in time and change in storage temperature are offered by way of example and are in no way limiting to the types on condition changes (e.g. time, exposure, technology type) to which the present inventions may be applied. In this example, the change in a nutritional property of potato salad is shown over a period of time including its preservation at the supermarket and a subsequent period of time including its local storage in a consumer's refrigerator, which is a local storage environment according to the present invention, and subsequent storage in the consumer's picnic cooler, which contains a local storage coupon according to the present invention. The graph shows that the potato salad is preserved at a first temperature, Temperature 1, for a first period of time indicated as 0 to 1, while at the supermarket. The potato salad is purchased by a consumer at time 1, and subsequently stored at a second temperature, Temperature 2, for a second period of time indicated as 1 to 2, during local storage in the consumer's refrigerator, which is a local storage environment according to the present invention. It is noted that Temperature 2 is greater than Temperature 1, and accordingly the shape of the graph changes at point A when the potato salad is taken from Temperature 1 and stored at Temperature 2. As in the preservation module, the local storage environment can identify the potato salad stored within it by reading or scanning its dynamic information identifier (or by the consumer entering it), can communicate with the nutritional substance information module, and accordingly can determine the potato salad's ΔN prior to placement within the refrigerator, and continue to track the potato salad's ΔN while in the refrigerator. The refrigerator is provided with a consumer interface, such as a screen, keyboard, sound system, or any known consumer interface. Alternatively, an application on the consumer's smartphone can enable the refrigerator to communicate with the smartphone such that the smartphone acts as the consumer interface. The consumer interface enables the refrigerator to communicate to the consumer that it contains the particular container of potato salad, information related to ΔN, including current nutritional, organoleptic, and aesthetic values of the potato salad while stored in the refrigerator. At time 2, the potato salad is taken from the refrigerator and placed inside the consumer's traditional picnic cooler, preferably along with an identifier such as a local storage coupon, a tag, barcode scan on the package, or any other suitable identifier (alternatively, an image recognition system can be used such that no separate identifier is needed) according to the present invention, where it is stored at Temperature 3, for a period of time indicated as 2 to 4. It is noted that Temperature 3 is greater than Temperature 2, and accordingly the shape of the graph changes at point B when the potato salad is taken from Temperature 2 and stored at Temperature 3. The local storage coupon can identify the potato salad stored within it by reading or scanning its dynamic information identifier (or by the consumer entering it), can communicate with the nutritional substance information module, and accordingly can determine the potato salad's ΔN prior to placement within the cooler, and continue to track the potato salad's ΔN while in the cooler. The coupon is provided with a consumer interface, such as a screen, keyboard, sound system, or any known consumer interface, or alternatively, an application on the consumer's smartphone can enable the coupon to communicate with the smartphone such that the smartphone acts as the consumer interface. The consumer interface enables the coupon to communicate to the consumer that the cooler contains the particular container of potato salad, information related to ΔN, including current nutritional, organoleptic, and aesthetic values of the potato salad while stored in the cooler, and when the potato salad will reach a minimum acceptable nutritional, organoleptic, or aesthetic value, indicated by “Minimum” on the vertical axis of the graph. The minimum acceptable values may be automatically provided by the information module, may be provided by the consumer through the consumer interface, or may be the higher of the two values. In this case the consumer can see how the nutritional value of the potato salad has degraded prior to placing it in the cooler with the coupon and can continue to see how the nutritional value degrades during local storage in the cooler, and when it will reach its minimum acceptable nutritional value. For example, at the time indicated as 3, the consumer can determine the residual nutritional value of the potato salad, corresponding to point C and “Residual” on the vertical axis of the graph. Further, the consumer can determine the potato salad's nutritional value will reach a minimum acceptable level at time 4, as indicated by “Minimum” on the vertical axis of the graph, thus knowing the window of time in which the potato salad in the cooler will maintain an acceptable nutritional level, as indicated by time 2 to 4. Further, the coupon can notify the consumer through the consumer interface when the potato salad's nutritional value has reached or fallen below the minimum acceptable value.

It is understood that local storage environments according to the present invention can comprise any local storage environment for a nutritional substance provided with the features enabling it to identify a dynamic information identifier on the nutritional substance, track one or more conditions related to a ΔN of the nutritional substance, communicate with the nutritional substance information module, determine a current ΔN, such as by the use of any known environmental or nutritional substance attribute sensor including a weight measurement sensor or scale, track and predict the ΔN of the nutritional substance while stored therein, and communicate information related to the ΔN to a consumer. In some embodiments, a standalone scale may be provided for removing the nutritional substance from the local storage environment and determining the weight of all or a portion of the nutritional substance in preparation for conditioning or consumption in order to determine the ΔN of the portion of nutritional substance removed from the local storage environment. Examples of such local storage environments include, but are not limited to: a pantry capable of identifying a dynamic information identifier on canned or bottled goods and tracking one or more conditions related to a ΔN of the canned or bottled goods, such as time, storage temperature, and weight; a shelf capable of identifying a dynamic information identifier on dry goods and tracking one or more conditions related to a ΔN of the dry goods, such as time, storage humidity and weight; a vegetable bin capable of identifying a dynamic information identifier on vegetables and tracking one or more conditions related to a ΔN of the vegetables, such as time, storage temperature, gaseous or volatile emissions from the vegetables, color of the vegetables, weight, and storage humidity; a drawer or storage container capable of identifying a dynamic information identifier on fruit and tracking one or more conditions related to a ΔN of the fruit, such as time, storage temperature, gaseous or volatile emissions from the fruit, weight, color of the fruit, and exposure to light; a medicine cabinet capable of identifying a dynamic information identifier on medicaments and tracking one or more conditions related to a ΔN of the medicaments, such as time, storage temperature, storage humidity, weight and exposure to light; a standalone scale capable of identifying a dynamic information identifier on a nutritional substance or optically identifying the substance itself and determining the weight of the nutritional substance in order to calculate a ΔN. These local storage environments or standalone scales may be provided with a consumer interface, such as a screen, keyboard, sound system, or any known consumer interface. Standalone scales include any freestanding electronic scale that is capable of detecting the weight of a nutritional substance and outputting that weight to the nutritional substance information module 100 or other components of the system to determine a ΔN and display that a ΔN to the consumer. In some embodiments weight or mass determination may be utilized by an optical object recognition system, in place of or in addition to a scale. For example, various products are available that are capable of using optical technology to visually identify produce and other nutritional substances, and various other items. For example, an automated optical fruit recognition system developed by Fraunhofer is capable of detecting and identifying various produce optically as described by an article titled “Automated Fruit Recognition” available at http://www.isob.fraunhofer.de/servlet/is/33328/ which is incorporated by reference herein in its entirety. Accordingly, a user could then utilize their mobile phone or other devices with optical sensors to identify nutritional substances. Additionally, an optical object recognition system is disclosed in U.S. Pat. No. 6,310,964 that is described as capable of detecting identity of produce and is incorporated herein by reference in its entirety.

An application on the consumer's smartphone can enable these local storage environments or standalone scales to communicate with the smartphone such that the smartphone acts as the consumer interface. The consumer interface enables the local storage environment or standalone scale to communicate to the consumer that it contains a particular nutritional substance, information related to its ΔN including a ΔN based on the weight of the nutritional substance, including current nutritional, organoleptic, and aesthetic values of the nutritional substance while stored in the local storage environment. In some embodiments, the local storage environment may be placed directly on the scale in order to determine the weight of the nutritional substance inside the local storage environment. These sensors may also measure thee 3-D dimensions of a nutritional substances to estimate its volume and weight.

It is understood that local storage containers according to the present invention can comprise any local storage container for a nutritional substance provided with the features enabling it to identify a dynamic information identifier on the nutritional substance, track one or more conditions related to a ΔN of the nutritional substance, communicate with the nutritional substance information module, determine a current ΔN, such as by the use of any known environmental or nutritional substance attribute sensor, track and predict the ΔN of the nutritional substance while stored therein, and communicate information related to the ΔN to a consumer. Examples of such local storage containers include, but are not limited to: a plastic, sealable container capable of identifying a dynamic information identifier on dry goods and tracking one or more conditions related to a ΔN of the dry goods, such as time, gaseous or volatile emissions from the dry goods, weight, color of the dry goods, and storage humidity; a tray capable of identifying a dynamic information identifier on fruit and tracking one or more conditions related to a ΔN of the fruit, such as time, gaseous or volatile emissions from the fruit, color of the fruit, weight, storage temperature, and exposure to light; a resealable bag capable of identifying a dynamic information identifier on vegetables and tracking one or more conditions related to a ΔN of the vegetables, such as time, storage temperature, gaseous or volatile emissions from the vegetables, color of the vegetables, and storage humidity; a purse capable of identifying a dynamic information identifier on a medicament and tracking one or more conditions related to a ΔN of the medicament, such as time, storage temperature, storage humidity, and exposure to light; a picnic cooler capable of identifying a dynamic information identifier on potato salad and tracking one or more conditions related to a ΔN of the potato salad, such as time, gaseous or volatile emissions from the potato salad, weight, color of the potato salad, and storage temperature. These local storage containers may be provided with a consumer interface, such as a screen, keyboard, sound system, or any known consumer interface. Alternatively, an application on the consumer's smartphone can enable these local storage containers to communicate with the smartphone such that the smartphone acts as the consumer interface. The consumer interface enables the local storage container to communicate to the consumer that it contains a particular nutritional substance, information related to its ΔN, including current nutritional, organoleptic, and aesthetic values of the nutritional substance while stored in the local storage container.

It is understood that local storage coupons according to the present invention can comprise any form of tag, badge, transponder, label, or any other device, individually and collectively referred to herein as a coupon, placed in proximity to a traditional local storage environment or traditional local storage container, and capable of identifying a dynamic information identifier on a nutritional substance stored in the traditional local storage environment or traditional local storage container, tracking one or more conditions related to a ΔN of the nutritional substance, communicating with the nutritional substance information module, determining a current ΔN, such as by the use of any known environmental or nutritional substance attribute sensor, tracking and predicting the ΔN of the nutritional substance, and communicating information related to the ΔN to a consumer. Examples of such local storage coupons include, but are not limited to: a coupon placed in a plastic container with dry goods, wherein the coupon is capable of identifying a dynamic information identifier on dry goods and tracking one or more conditions related to a ΔN of the dry goods, such as time, gaseous or volatile emissions from the dry goods, color of the dry goods, weight, and storage humidity; a coupon placed on a tray for holding fruit, wherein the coupon is capable of identifying a dynamic information identifier on fruit and tracking one or more conditions related to a ΔN of the fruit, such as time, storage temperature, gaseous or volatile emissions from the fruit, color of the fruit, and exposure to light; a coupon placed within a resealable vegetable bag, wherein the coupon is capable of identifying a dynamic information identifier on vegetables and tracking one or more conditions related to a ΔN of the vegetables, such as time, storage temperature, gaseous or volatile emissions from the vegetables, weight, color of the vegetables, and storage humidity; a coupon placed within a purse, wherein the coupon is capable of identifying a dynamic information identifier on a medicament placed within the purse and tracking one or more conditions related to a ΔN of the medicament, such as time, storage temperature, storage humidity, and exposure to light; a coupon attached to the inner surface of a picnic cooler, wherein the coupon is capable of identifying a dynamic information identifier on potato salad stored in the cooler and tracking one or more conditions related to a ΔN of the potato salad, such as time, gaseous or volatile emissions from the potato salad, color of the potato salad, and storage temperature; a coupon hung in a pantry, wherein the coupon is capable of identifying a dynamic information identifier on canned or bottled goods and tracking one or more conditions related to a ΔN of the canned or bottled goods, such as time, exposure to light (in the case of bottled goods), and storage temperature; a coupon attached to a shelf, wherein the coupon is capable of identifying a dynamic information identifier on dry goods and tracking one or more conditions related to a ΔN of the dry goods, such as time, gaseous or volatile emissions from the dry goods, color of the dry goods, weight, and storage humidity; a coupon attached to an inner surface of a vegetable bin, wherein the coupon is capable of identifying a dynamic information identifier on vegetables and tracking one or more conditions related to a ΔN of the vegetables, such as time, gaseous or volatile emissions from the vegetables, weight, color of the vegetables, storage temperature, and storage humidity; a coupon placed within a drawer, wherein the coupon is capable of identifying a dynamic information identifier on fruit and tracking one or more conditions related to a ΔN of the fruit, such as time, gaseous or volatile emissions from the fruit, color of the fruit, storage temperature, and exposure to light; a coupon attached to the inner surface of a medicine cabinet, wherein the coupon is capable of identifying a dynamic information identifier on medicaments and track one or more conditions related to a ΔN of the medicaments, such as time, storage temperature, storage humidity, and exposure to light.

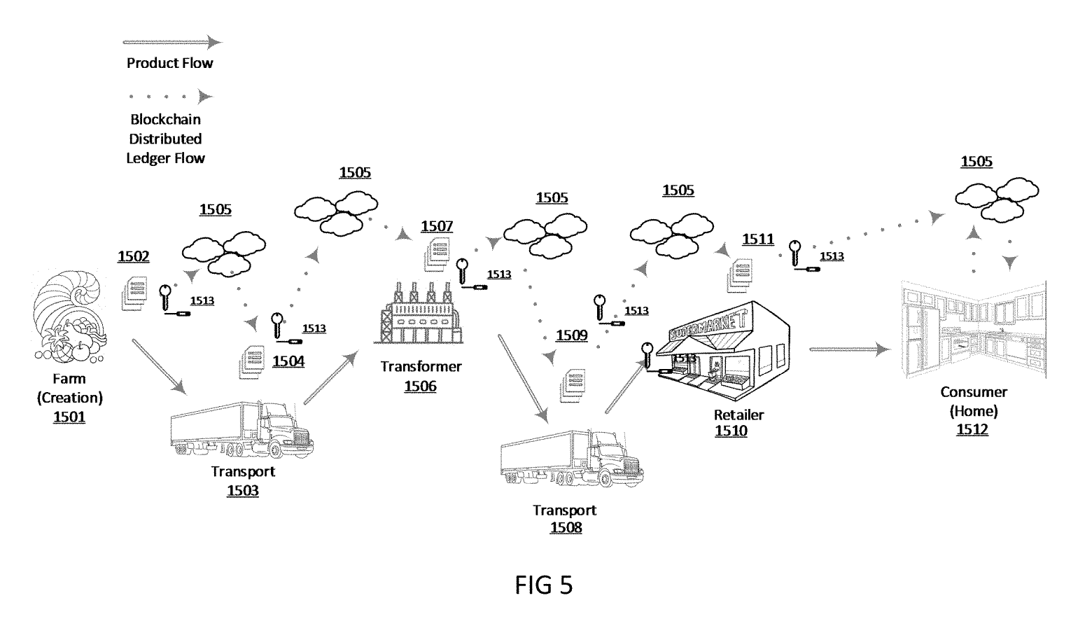

FIG. 5 illustrates the use of block chain distributed ledgers to alert consumers of products purchased and stored at the household. The creation of the blockchain distributed ledger can start at the farm 1501, or at any other point in the supply chain, such as transport 1503 and 1508, transformer 1506, or retailer 1508, depending on which are authorized participants in the supply chain. As the nutritional substances moves from one module to another (e.g. creation, preservation, transformation, consumption), each authorized participant creates transactions the network 1505 that are encrypted by a private key 1513, but it is not possible to view and alter previous transactions without the execution of a consensus protocol between each participant or node. The consensus protocols determine whether a transaction should be validated in the ledger (1502, 1504, 1507, 1509 and 1511). Some consensus algorithms allow private setups in the network, such as Proof-of-Authority (PoA), developed by Applicature. It uses the concept of “validators”, preapproved authority nodes that can approve and validate transactions and blocks. Validators can approve new nodes by running software allowing the addition of transactions in a given block or blocks. A validator is incentivized to uphold the transaction process, as their identities can be attached to a negative reputation.

In this example, as the nutritional substance moves through the supply chain where it is transported, transformed, transported, sold and consumer, a distributed ledger is created by validators, where ΔN data (measured through attribute sensors or calculated) is added to the ledger in subsequent nodes. The nutritional substance attributes sensors can beneficially be provided with, or combined with, other nutritional substance modules, including transformation, preservation, and consumer modules. Furthermore, the nutritional substance attribute sensors could be provided with the local storage environments, containers, and coupons described herein. Nutritional substance attribute sensors, or at least a portion of the nutritional substance attribute sensor, could be provided with or incorporated into the package of any prepackaged nutritional substance, such that the consumer or appliance sensor may interrogate the package without disrupting its integrity to obtain information related to a nutritional, organoleptic, or aesthetic value of the nutritional substance contained therein.

As the nutritional substance reaches the consumer household containing smart appliances 1512, the sensors in the appliance can detect the nutritional substance by reading or scanning information on the package or identifying the nutritional substance by other means such as machine vision, hyperspectral imaging, chemical sensing (aroma, gas), or genetic detection, amongst others. Further, nutritional substance attribute sensors, or at least a portion of the nutritional substance attribute sensor, could be provided coupled with, or incorporated into smartphones. This would enable a wide array of users and scenarios wherein nutritional substances can be identified and the current nutritional, organoleptic, and aesthetic state can be determined. The attribute sensors are connected to the information module or blockchain cloud server 1505 via internet in such a way that the attribute sensors can communicate with or receive communications with blockchain cloud server. For instance, the sensors can identify and send this information of one or more nutritional substances that are stored in the household (local storage or conditioner). The blockchain server in turn provides ΔN information on the nutritional substances available in the household in communication with an app in either the smart appliance or the mobile device. In the case of a recall of a production batch of one or more nutritional substances, the system can verify if any of the recalled nutritional substances are in the household, and if so, send an alert, notification or alarm to the app. In the case that the nutritional substance has to be recalled, the blockchain cloud server will send information to consumer indicating how to dispose of the product, requesting confirmation from the user that the nutritional substance was disposed to minimize the risk to the consumers in the household, to manage liability, and to update the outstanding risk profile of a contamination.

Unless the context clearly requires otherwise, throughout the description and the claims, the words “comprise,” “comprising,” and the like are to be construed in an inclusive sense (i.e., to say, in the sense of “including, but not limited to”), as opposed to an exclusive or exhaustive sense. As used herein, the terms “connected,” “coupled,” or any variant thereof means any connection or coupling, either direct or indirect, between two or more elements. Such a coupling or connection between the elements can be physical, logical, or a combination thereof. Additionally, the words “herein,” “above,” “below,” and words of similar import, when used in this application, refer to this application as a whole and not to any particular portions of this application. Where the context permits, words in the above Detailed Description using the singular or plural number may also include the plural or singular number respectively. The word “or,” in reference to a list of two or more items, covers all of the following interpretations of the word: any of the items in the list, all of the items in the list, and any combination of the items in the list.

The above Detailed Description of examples of the invention is not intended to be exhaustive or to limit the invention to the precise form disclosed above. While specific examples for the invention are described above for illustrative purposes, various equivalent modifications are possible within the scope of the invention, as those skilled in the relevant art will recognize. While processes or blocks are presented in a given order in this application, alternative implementations may perform routines having steps performed in a different order, or employ systems having blocks in a different order. Some processes or blocks may be deleted, moved, added, subdivided, combined, and/or modified to provide alternative or sub-combinations. Also, while processes or blocks are at times shown as being performed in series, these processes or blocks may instead be performed or implemented in parallel, or may be performed at different times. Further any specific numbers noted herein are only examples. It is understood that alternative implementations may employ differing values or ranges.

The various illustrations and teachings provided herein can also be applied to systems other than the system described above. The elements and acts of the various examples described above can be combined to provide further implementations of the invention.

Any patents and applications and other references noted above, including any that may be listed in accompanying filing papers, are incorporated herein by reference. Aspects of the invention can be modified, if necessary, to employ the systems, functions, and concepts included in such references to provide further implementations of the invention.

These and other changes can be made to the invention in light of the above Detailed Description. While the above description describes certain examples of the invention, and describes the best mode contemplated, no matter how detailed the above appears in text, the invention can be practiced in many ways. Details of the system may vary considerably in its specific implementation, while still being encompassed by the invention disclosed herein. As noted above, particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific examples disclosed in the specification, unless the above Detailed Description section explicitly defines such terms. Accordingly, the actual scope of the invention encompasses not only the disclosed examples, but also all equivalent ways of practicing or implementing the invention under the claims.

While certain aspects of the invention are presented below in certain claim forms, the applicant contemplates the various aspects of the invention in any number of claim forms. For example, while only one aspect of the invention is recited as a means-plus-function claim under 35 U.S.C. § 112, sixth paragraph, other aspects may likewise be embodied as a means-plus-function claim, or in other forms, such as being embodied in a computer-readable medium. Any claims intended to be treated under 35 U.S.C. § 112, ¶6 will begin with the words “means for.” Accordingly, the applicant reserves the right to add additional claims after filing the application to pursue such additional claim forms for other aspects of the invention.

We claim:

1. A food traceability system, comprising:
sensors that measure data associated with a nutritional substance or food handling environment at any stage of a food supply chain;
a distributed ledger or database, with data stored in three or more locations, configured to store the sensor measurements associated with the nutritional substance or food handling environment for storage, processing, conditioning, or retail;
a processor configured to calculate the status of the nutritional substances based on the sensor data;
a database configured to associate status and ownership of the nutritional substance with various parties in the supply chain, including the end consumer who has purchased the item; and
a cryptographic mechanism configured to verify the authenticity and source of data associated with a nutritional substance or food handling environment, and to provide a consensus mechanism for determining the valid value of a data element.

2. The food traceability system of claim 1 wherein the processor is co-located with any one stage of the food supply system.

3. The food traceability system of claim 1 wherein the processor is hosted in a cloud based system.

4. The food traceability system of claim 1 wherein the cryptographic mechanism is comprised of one of more of: blockchain, Merkle trees or other cryptographically signed data structures.

5. The food traceability system of claim 1 further comprising: a reporting mechanism configured to access the database to provide information on the nutritional substance.

6. The food traceability system of claim 5 wherein the reporting mechanism is comprised of any one of more of: email, SMS, Web page, application, or embedded appliance software functionality.

7. The food traceability system of claim 5 wherein the information is based on at least two measurements of the item.

8. The food traceability system of claim 1 further comprising: an alerting network, configured to communicate with supply chain participants or consumers, to identify nutritional substance items and associated ΔN, organoleptic status, or food-safety status.

9. A consumer sensor system, comprised of: a mobile device, smart appliance, or modular sensor node configured to identify nutritional substances by a dynamic information identifier, bar-code, identification number, optical recognition, computer vision, spectral recognition, electronic recognition such as RFID, NFC, or any other electromagnetic tag, product packaging or labels, or molecular composition of a nutritional substance or associated additives.

10. A consumer sensor system, comprised of: a mobile device, smart appliance, or modular sensor node configured to sense ΔN of food substances, including food safety, freshness or organoleptic status.

11. The consumer sensor system of claim 10 wherein the sensing ΔN of food substances is accomplished by any one or more of: optical recognition, computer vision, spectral recognition, gas sensing, electronic recognition such as RFID, NFC, or any other electromagnetic tag, active product packaging or labels, sensors for reading packages or electromagnetic tags, or molecular composition of a nutritional substance or associated additives.

12. The consumer sensor system of claim 11 wherein electronic receipts or customer loyalty programs are used to determine which consumers have purchased a particular nutritional substance.
